(19) United States
(12) Patent Application Publication
(10) Pub. No.: US 2022/0160299 A1
(43) Pub. Date: May 26, 2022

(54) MOTION CAPTURE SYSTEM

(71) Applicant: RLT IP LTD., Gosport (GB)

(72) Inventors: David M. Jessop, New Alresford (GB); Peter Robins, Woolton Hill (GB); Jonathan M. Dalzell, Lee-On-The-Solent (GB); Daniel A. Frost, Gosport (GB); Richard E. Collins, Newport (GB); Chad H. Saunders, Chichester (GB)

(21) Appl. No.: 17/623,173

(22) PCT Filed: Jun. 28, 2019
(86) PCT No.: PCT/EP2019/067486

§ 371 Date: Dec. 27, 2021

(51) Int. Cl. A61B 5/00; A61B 5/11; G09B 19/00

(52) U.S. Cl. CPC A61B 5/6804; A61B 5/11; A61B 2562/16; A61B 2503/10; G09B 19/0038

(57) ABSTRACT

A motion capture suit includes a top or pants, a sensor socket fixed to a portion of the top or the pants about a limb, and a sensor inserted in the sensor socket. The sensor includes a measurement device having measurement axes that are offset by about 45 degrees from a long axis of the limb.

### BACKGROUND

Wearable technologies are electronic devices incorporated into clothing or worn on the body. They are often used to monitor an athlete's movements to improve performance. Wearable devices may include motion sensors such as accelerometers and gyroscopes. They may also include physiologic sensors such as heart rate monitors and temperature sensors.

### BRIEF DESCRIPTION OF THE DRAWINGS

In the drawings:

FIG. 1 is a block diagram of a motion sensing system in some examples of the present disclosure.

FIG. 2 illustrates a skeletal model for generating the avatar in some examples of the present disclosure.

FIG. 3 illustrates a motion capture suit of FIG. 1 in some examples of the present disclosure.

FIG. 4 illustrates a sensor socket fixed to a location on the motion capture suit of FIG. 1 in some examples of the present disclosure.

FIG. 5 illustrates a sensor just before being inserted in sensor socket in some examples of the present disclosure.

FIG. 6 illustrates a sensor inserted and locked to a sensor socket in some examples of the present disclosure.

FIG. 7 illustrates a sensor set in some examples of the present disclosure.

FIG. 8 illustrates the orientation of a sensor with a gyroscope having a specific orientation for a given activity in some examples of the present disclosure.

FIG. 9 illustrates a sensor socket with a socket base fixed to a motion capture suit and a rotatable bezel around the socket base in some examples of the present disclosure.

FIG. 10 is a block diagram illustrating a sensor in some examples of the present disclosure.

FIG. 11 is a swimlane diagram demonstrating how a pod of FIG. 1 is configured in some examples of the present disclosure.

FIG. 12 is a flowchart of a method illustrating the operations of the pod of FIG. 1 in some examples of the present disclosure.

Use of the same reference numbers in different figures indicates similar or identical elements.

### DETAILED DESCRIPTION

In some examples of the present disclosure, a motion sensing system is provided for full or partial body motion capture and reconstruction in a variety of circumstances and environments. The system includes a motion capture suit with sensors located at strategic locations on a user's body. A sensor may be oriented to have its axes of measurement offset from an axis of the great motion (e.g., acceleration or angular velocity) in order to avoid topping out or saturating the sensor. On the motion capture suit, a sensor socket may be rotatable so an inserted sensor's axes of measurement may be adjusted for a particular activity.

Additional sensors may be added to the system for tracking a single body part in greater detail or an extra piece of equipment, such as a golf club. Different types of sensors may be added to the system, such as pressure insoles, heart rate monitors, etc. Sensors with different specifications may be placed in different locations of the body, thereby allowing the same suit to be used for different activities or users of different capabilities.

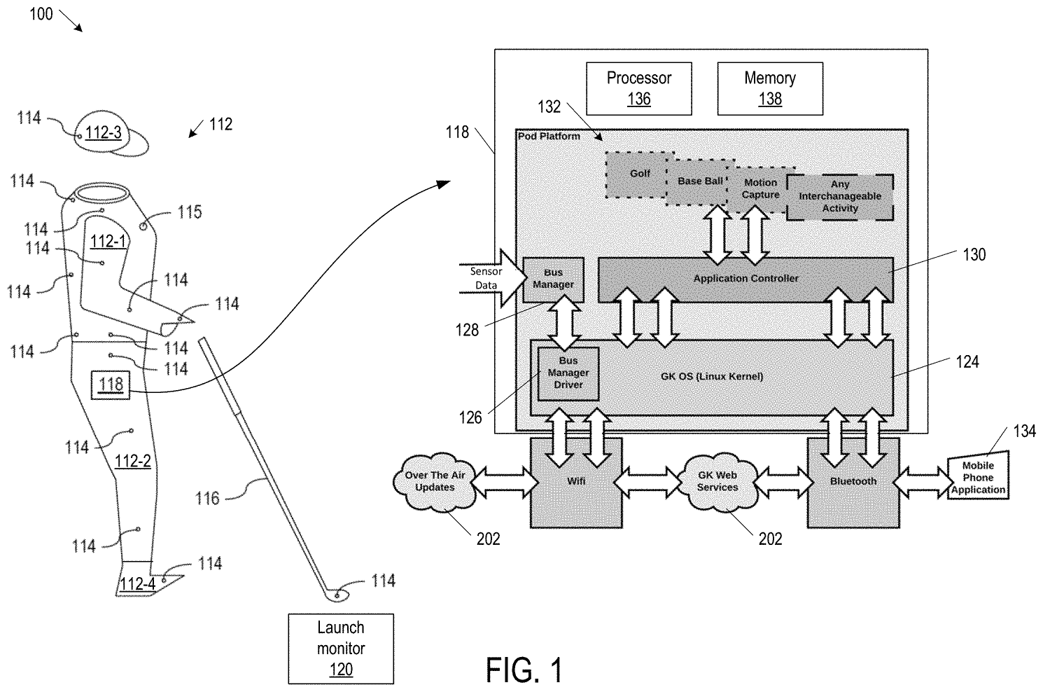

FIG. 1 is a block diagram of a motion sensing system 100 in some examples of the present disclosure. Motion sensing system 100 is customizable and configurable according to the action undertaken by a user, as well as the profile and the biometrics of the user.

Motion sensing system 100 includes a motion capture suit 112 with motion tracking sensors 114 that directly measure movements of the user wearing suit 112 during an activity to be analyzed. Motion capture suit 112 may be one piece or may include multiple sections, such as an upper body section or shirt 112-1, a lower body section or pants 112-2, a cap or hood 112-3, and socks or insoles 112-4. Motion capture suit 112 may be elastic and relatively tight fitting to minimize shifting of motion tracking sensors 114 relative to the body. Motion tracking sensors 114 are located in or on motion capture suit 112 to measure movement of specific body parts. Each motion tracking sensor 114 may include a combination of accelerometer, gyroscope, and magnetometer, whose raw data outputs may be processed to determine the position, orientation, and movement of the corresponding body part. The raw data outputs include accelerations (m/s²) along the measurement axes of the accelerometer, angular velocity (rad/s) about the measurement axes of the gyroscope, and magnetic field vector components (Gauss) along the measurement axes of the magnetometer.

Motion sensing system 100 may include one or more biofeedback sensors 115 that measure physiological functions or attributes of the subject such as the user's heart rate, respiration, blood pressure, or body temperature. For example, a pressure insole in the user's shoe can measure the timing and amount of weight transfer from one foot to another or between the ball and toe of the subject's foot during the measured activity.

The user may employ a piece of equipment 116 during the measured activity. Equipment 116 may, for example, be sporting equipment such as a golf club, a tennis or badminton racket, a hockey stick, a baseball or cricket bat, a ball, a puck, or a shuttle that the subject uses during a measured sports activity. Equipment 116 may alternatively be a tool, exercise equipment, a crutch, a prosthetic, or any item that a subject is being trained to use. One or more motion tracking sensors 114 may be attached to equipment 116 to determine the position, orientation, movement, or bend/flex of equipment 116.

Motion sensing system 100 may include an electronic device 120 that provides additional motion data of the subject or equipment 116, such as a golf launch monitor that provides swing speed, ball speed, and ball spin rate.

A sensor controller 118, also known as a “pod,” is attached to or carried in motion capture suit 112. Pod 118 has wired or wireless connections to sensors 114 and 115. Pod 118 processes the raw motion data from sensors 114 to produce geometric data for a skeletal model and metrics calculated from a combination of the raw motion data and the geometric data. Pod 118 transmits the geometric data, the metrics, and the biofeedback data to an app 134 on a smart device via wireless or wired connection (e.g., Bluetooth) during or after the measured activity. The smart device may be a smart phone, a tablet computer, a laptop computer, or a desktop computer. App 134 generates scores from the geometric data and provides visual feedback in the form of an avatar that shows the movement of the user.

For hardware, pod 118 includes processor 136 and memory 138 for executing and storing the software. Pod 118 also includes a RS-485 data bus transceiver (not shown) for communicating with sensors 114 and 115, a Wi-Fi transceiver (not shown) for communicating with a wireless network to access the Internet, and a Bluetooth transceiver (not shown) for communicating with app 134 on the smart device.

For software, pod 118 includes an operating system (OS) 124 with a bus manager driver 126 executed by the processor, a bus manager 128 executed by the data bus transceiver, and an application controller 130 that runs on the OS, and a number of activity executables 132 that detect and analyze actions of different activities (e.g., a golf swing for golf, a bat swing for baseball, and a groundstroke for tennis). Pod 118 and any power source may be stored in a pocket of motion capture suit 112.

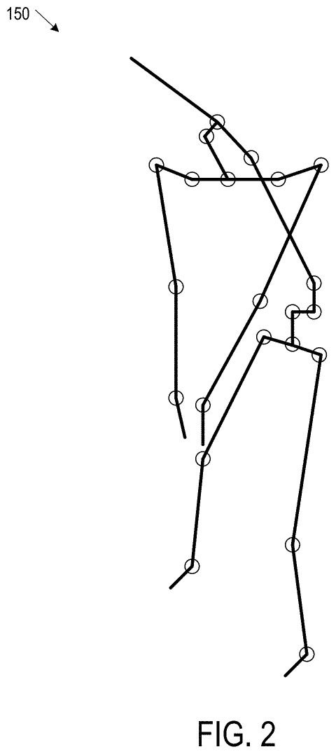

FIG. 2 illustrates a skeletal model 150 for generating the avatar in some examples of the present disclosure. Skeletal model 150 is a hierarchical set of joint nodes and limb segments that link the joint nodes. Each limb segment represents a bone, or a fixed bone group, in the human body. The highest joint node in the hierarchy is the root node. In a chain of joint nodes, the joint nodes closer to the root node are higher in the hierarchy than joint nodes further from the root node. Movements of skeletal model 150 are represented by movements of the joint nodes. Assuming the limb segments are rigid bodies, movement of any joint node is represented by a translation (e.g., a vector) and a rotation (e.g., a quaternion) of the joint node, where the rotation of the joint node determines the orientation of the limb segment extending from the joint node. Movement of the root node controls the position and orientation of the skeleton model in a three-dimensional space. Movement of any other joint node in the hierarchy is relative to that node's parent. In particular, all of the descendant joint nodes from the root node form an articulated chain, where the coordinate frame of a child node is always relative to the coordinate frame of its parent node.

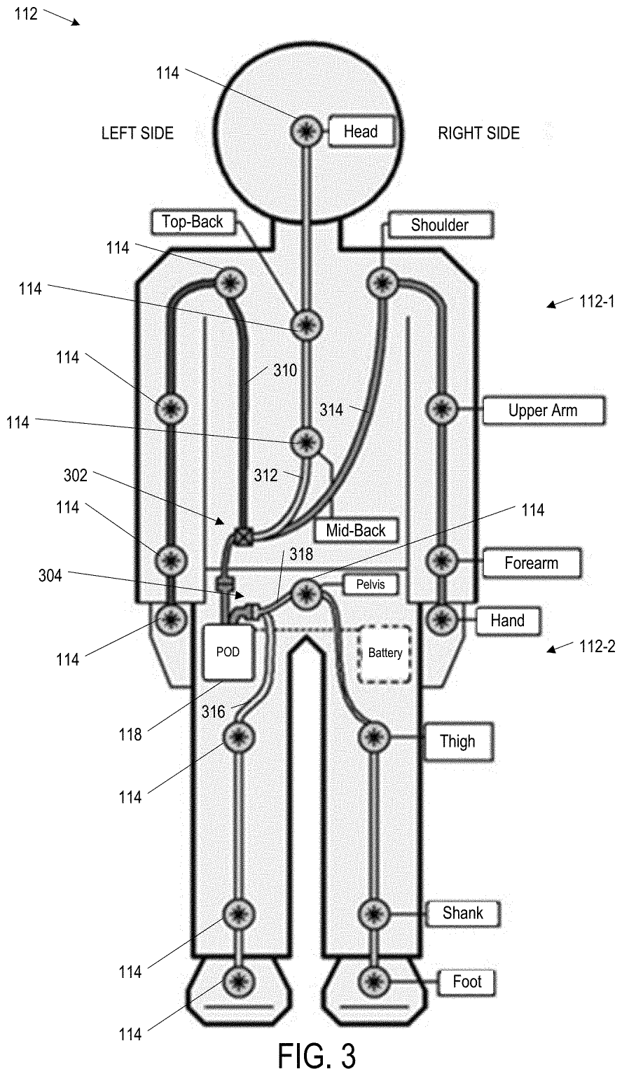

FIG. 3 illustrates motion capture suit 112 in some examples of the present disclosure. Suit 112 may be made of 75% nylon and 25% elastane to provide a compression fit that reduces wobble from the human body. Suit 112 includes multiple sensor networks and each sensor network supports a number of sensors 114. Thus, pod 118 (FIG. 1) can configure motion capture suit 112 to use selected networks or even selected sensors. Sensors 114 can be added and removed from each sensor network. For example, a low range sensor 114 on a leg may be replaced with a high range sensor 114 on a hand because the focus is on the lower body for a kicking action. Further, if there is great interest in the movement of the spine, additional sensors 114 may be added to this area.

In some examples, motion capture suit 112 includes (1) a top 112-1 with an upper wiring harness 302 and (2) pants 112-2 with a lower wiring harness 304. Top 112-1 and pants 112-2 may be joined by a zipper or snap fasteners to prevent the garment from riding up or down. Upper wiring harness 302 and lower wiring harness 304 are connected to pod 118, which is held in a pocket of motion capture suit 112.

Upper wiring harness 302 includes three sensor networks 310, 312, and 314. Lower wiring harness 304 includes two sensor networks 316 and 318. Each sensor network is a chain of sensors 114. The cables linking sensor sockets 402 are flexible and the lengths of each section of the cables are tailored according to the size of the suit/user. Additional slack (in the form of loops) may be added into the wires within the cables so that any pulling of a cable is absorbed by the wires and not by a sensor 114.

Sensors 114 are inertial measurement unit (IMU) sensors. Sensors 114 are placed at strategic locations over the body to track each major limb segment while minimizing movements due to contractions of underlying muscle mass. For each limb segment, the corresponding sensor 114 is generally located near the distal end and on the outer surface of the limb segment.

Five sensors 114 are placed to track the movements of the pelvis, mid-back, and upper back, left shoulder, and right shoulder. Sensors 114 are also located to track the movements of the head and feet. For example, sensors 114 may be equipped with hooks to loop over or into a hat and shoes. Approximate proximal-distal locations of sensors 114 are provided Table 1 below.

TABLE 1
Approximate sensor location on
each segment

| | | % location on |
|---|---|---|
| | | long axis of |
| Node Name | Description | limb segment |
| Upper Back | | 6 |
| L_Arm | | 78 |
| L_Foot | | 58 |
| L_Forearm | | 82 |
| L_Shank | | 77 |
| L_Thigh | | 84 |
| L_Hand | | 65 |
| L_Shoulder | L_Scap_Sho | 22 |
| | L_Scap_Spi | 27 |
| Pelvis | | 99 |
| R_Arm | | 75 |
| R_Foot | | 56 |
| R_Forearm | | 84 |
| R_Shank | | 77 |
| R_Thigh | | 80 |
| R_Hand | | 71 |
| R_Shoulder | R_Scap_Sho | 22 |
| | R_Scap_Spi | 27 |
| Mid-Back | | 58 |

In Table 1, percentages are expressed from proximal to distal end of the corresponding limb segment. For example, L_Arm=60 means that sensor 114 on the left arm is 60% along the length of the upper arm towards the elbow. Sensors 114 for the upper and mid-backs are expressed relative to the length of the length of the spine from top to bottom (e.g., from the cervical vertebrae 7 to the sacrum). Sensor 114 for the left shoulder is located based on L_Scap_Sho and L_Scap_Spi, where L_Scap_Sho is 22% along the imaginary line between the shoulder joints, and L_Scap_Spi is 27% along the length of the spine from top to bottom. Sensor 114 for the pelvis is located 99% along the length of the spine from top to bottom.

Sensors 114 are inserted into sensor sockets so the sensors can be easily removed and replaced. FIG. 4 illustrates a sensor socket 402 fixed to a location on suit 112 in some examples of the present disclosure. A hole in the material of suit 112 is provided for each sensor socket 402. The hole is reinforced with a polyurethane ring 403, which sensor socket 402 clamps onto to prevent fabric fraying and movement of sensor 114. While some sensor sockets 402 are fixed to suit 112, others may be equipped with clips for attachment to hat, shoe, or another apparel. Sensor socket 402 is daisy chained to other sensor sockets in a sensor network by cables within suit 112.

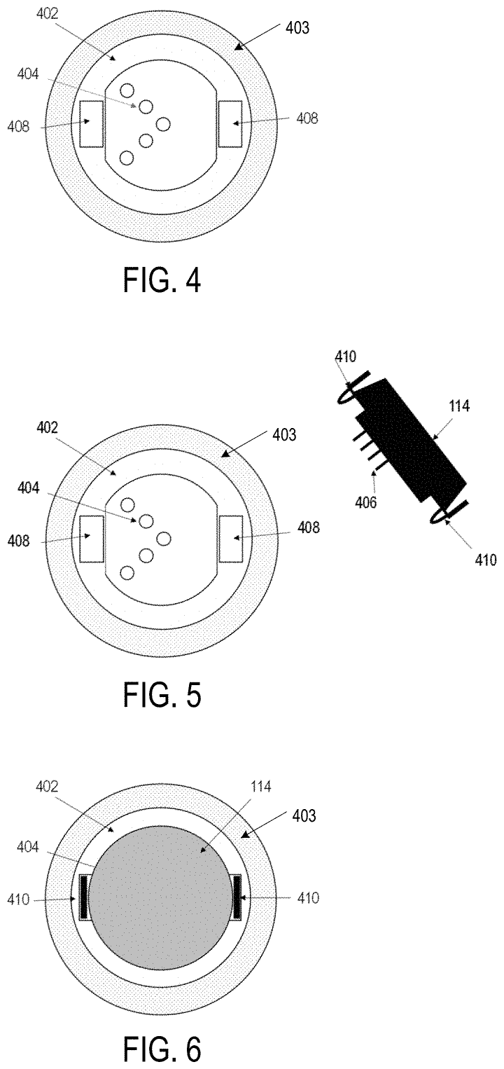

FIG. 5 illustrates a sensor 114 just before being inserted in sensor socket 402 in some examples of the present disclosure. Sensor socket 402 has a particular arrangement of contact sockets 404 for receiving contact pins 406 on sensor 114 so the sensor can only be inserted in the correct orientation into the sensor socket. For example, contact sockets 404 and contact pins 406 may be arranged in a “V” shape. Sensor socket 402 further includes holes 408 for receiving cantilever hooks 410 on sensor 114 that lock the sensor to the sensor socket. FIG. 6 illustrates sensor 114 inserted and locked to sensor socket 402 in some examples of the present disclosure. The release sensor 114 from sensor socket 402, the user squeezes the ends of hooks 410 and then pull the sensor out from the sensor socket.

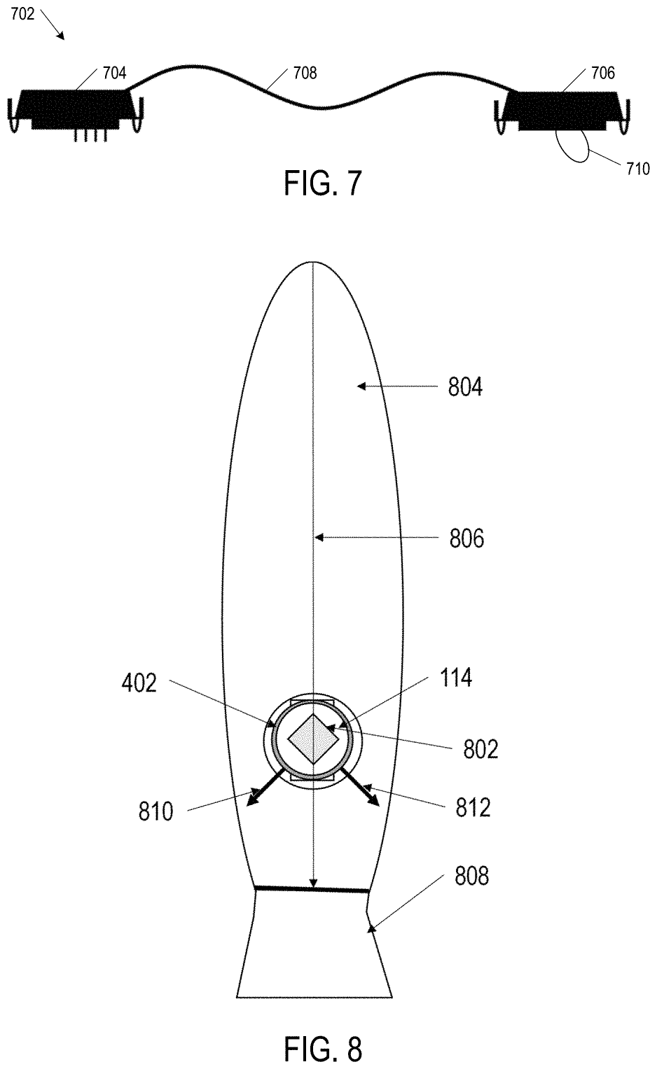

When it is desirable to extend a sensor network or add an additional sensor 114 about a location, a single sensor 114 may be replaced by a sensor set. FIG. 7 illustrates a sensor set 702 in some examples of the present disclosure. Sensor set 702 includes a base sensor 704 and an extended sensor 706. Base sensor 704 is configured like sensor 114 to fit in sensor socket 402 at the end of the sensor network. Base sensor 704 has a cable 708 that runs to extended sensor 706. Alternatively, sensors 704 and 706 include wireless transceivers that allow them to communicate wirelessly (e.g., Bluetooth). Extended sensor 706 may have an elastic loop or clip 710 to fix the sensor on another body part (e.g. fingers) or a piece of equipment (e.g., a golf club).

The interchangeability of sensors 114 means that the internal characteristics of the sensors may be altered or adapted according to the activity. It is possible that sensors 114 may “top out” or saturate. For example, the gyroscope in a sensor 114 has a maximum range of +/−4000 deg/s for each individual measurement axis (X, Y, Z) but some activities may go above this. In order to make the most of this range, sensor 114 may be placed so that the measurement axes of the gyroscope are aligned at 45 degrees to the axis of the segment about which it rotates fastest. For example, in golf, the hands are rotating fastest when the knuckles point downwards towards the ball around the point of contact. If the axis of rotation of the hands aligns directly with one of the measurement axes of the gyroscope in sensor 114 at this point, the limit of measurement is 4000 deg/s before saturation. However, if the orientation of sensor 114 is such that two of the measurement axes of the gyroscope are at 45 degrees to the axis of rotation, the maximum reading before saturation occurs is increased to 5,656.85 deg/s, which is an increase of 41%. If the measurement all three axes of the gyroscope are orientated so they were equally and maximally different to the rotation axis at maximum speed, the maximum measurement reaches 6,928.20 before saturation, which is an increase of 73%.

FIG. 8 illustrates the orientation of a sensor 114 with a gyroscope 802 having a specific orientation for a given activity in some examples of the present disclosure. Assume sensor 114 is located on a (forearm) limb segment 804 and the long axis 806 of the forearm experiences the greatest angular velocity for the give activity. As described before, sensor 114 is located 82 or 84% along the long axis 806 of forearm 804 toward (hand) limb segment 808. To avoid saturation, gyroscope 802 is oriented so at least two of its measurement axes (e.g., two measurement axes 810 and 812) are rotated about 45 degrees (e.g., within 5 to 15 degrees) relative to the axis of the greatest rotation when sensor 114 is inserted in sensor socket 402. In most activities, the axis of greatest rotation is the long axis 806. In some examples, measurement axes 810 and 812 are located in substantially the same plane as long axis 806 (e.g., within 5 to 15 degrees). When present, the accelerometer and the magnetometer may also be oriented in sensor 114 so their measurement axes are offset from the axes of the greatest corresponding measurements.

Alternatively, sensor 114 or sensor socket 402 may include a mechanism that allows the sensor to be rotated during adjustment but then fixed during use so as not to introduce artifacts into the measurement signal. Such a mechanism allows sensors 114 to be aligned to a given axis or segment. This functionality may be particularly useful when attaching sensors 14 to a piece of equipment such as a golf club as the sensor can be aligned to the long axis of the shaft and the face of the head.

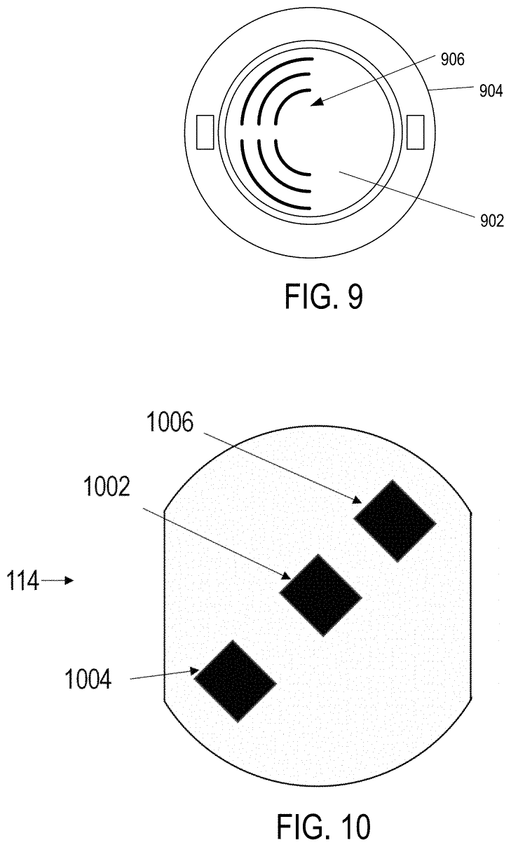

FIG. 9 illustrates a sensor socket 900 with a socket base 902 fixed to motion capture suit 112 and a rotatable bezel 904 around the socket base in some examples of the present disclosure. Rotatable bezel 904 has holes 402 for receiving cantilever hooks 410 of sensor 114 so the sensor is attachable to the bezel. Socket base 902 has contact arcs 906 for electrical connection with contact pins 406 of sensor 114. When secured to rotatable bezel 904, sensor 114 can be rotated and locked (e.g., by a spring-loaded pin) in one (1) degree increments to provide a shift from −90 to +90 degrees of the measurement axes of sensor 114. Alternatively, rotatable bezel 904 is fixed to motion capture suit 112 and socket base 902 is rotatable relative to the bezel. This allows socket base 902 to be implemented with pin contact sockets 404 instead of contact arcs 906 for electrical connection with contact pins 406 of sensor 114.

App 134 may determine the angle at which to set the orientation of sensor 114 to the user based on measurements taken from previous performances recorded during use of motion capture suit 112. Alternatively, app 134 may provide the angle based on knowledge of the skill/technique/variation that the user is about to perform. In other words, for certain sports, app 134 may have predetermined angle for sensor 114 based on statistical data.

When sensors 114 are swapped to a different location, pod 118 is able to recognize this due to each sensor having a unique ID. As the calibration data and offsets for each sensor and its components (accelerometer, magnetometer, and gyroscope) are stored both remotely and locally, pod 118 can correctly associate this data with the owner sensor 114 no matter its location in motion capture suit 112.

While IMU type sensors 114 are discussed in the present disclosure, motion system 100 (FIG. 1) is not limited only to this type of sensor. Due to sensors 114 being removable from motion capture suit 112 (FIGS. 1 and 3), they may be swapped with ones that incorporate additional functionality in order to suit the activity, purpose, or environment as required.

FIG. 10 is a block diagram illustrating sensor 114 in some examples of the present disclosure. Sensor 114 includes a 6-axis accelerometer/gyroscope 1002, a 3-axis magnetometer 1004, and an optional 3-axis high G accelerometer 1006. Sensor 114 may include additional components. In some examples, sensor 114 includes a microphone incorporate voice command and communication functions. In some examples, sensor 114 includes a speaker to provide audio feedback, communication functions, and music playback. In some examples, sensor 114 includes a light to provide visual feedback. In some examples, sensor 114 includes a screen to display information and feedback. In some examples, sensor 114 includes an interactive display for displaying interactive feedback. In some examples, sensor 114 includes a mobile communication device such as a mobile phone, music player, or a payment device. In some examples, sensor 114 includes a proximity sensor to detect the presence of other users. In some examples, sensor 114 includes haptic feedback mechanisms to provide haptic feedback. In some examples, sensor 114 includes a medication dispenser for administering medication. In some examples, sensor 114 includes an environment sensor that detects temperature, humidity, wind speed, air quality, or altitude. In some examples, sensor 114 includes a hazards sensor that detects smoke, carbon monoxide, or other harmful gases. In some examples, sensor 114 includes an impact or shear force sensor.

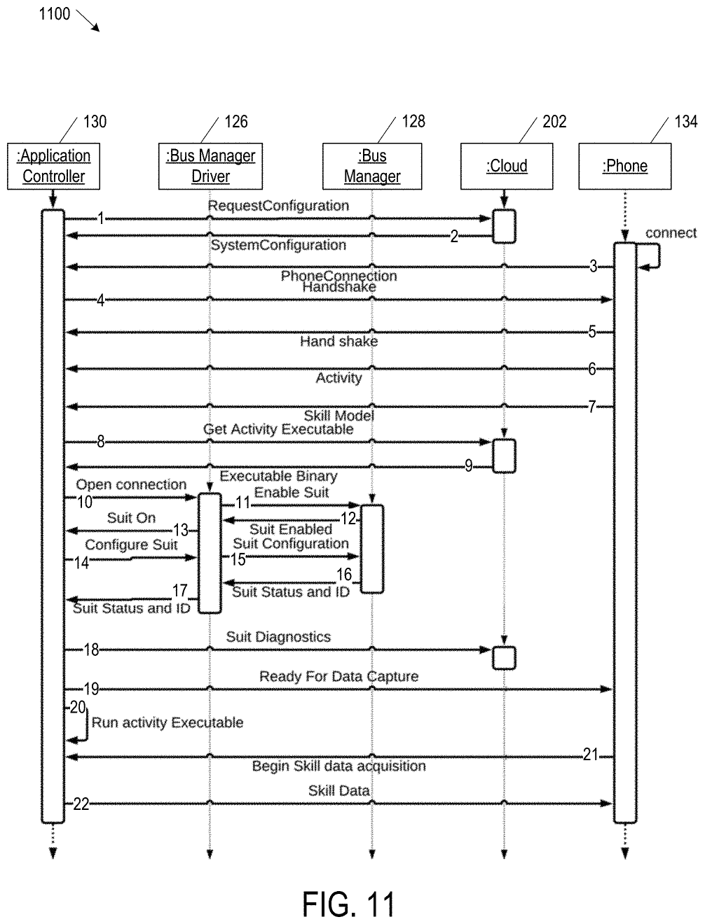

FIG. 11 is a swimlane diagram demonstrating how pod 118 is configured in some examples of the present disclosure. In step 1, upon startup, application controller 130 requests for the system configuration of pod 118 from a provider 202 of motion sensing system 100 over the Internet. The system configuration identifies sensors, activity executables, and other hardware and software components that the user is authorized to use (e.g., by subscription). Application controller 130 may connect to the Internet through the Wi-Fi or Bluetooth. In step 2, provider 202 sends the system configuration to application 130 to verify and enable the authorized hardware and software for the user.

In step 3, an interactive app 134 (FIG. 1) on a smart device requests to connect with application controller 130 over Bluetooth. The smart device may be a laptop, a smart phone, or a tablet. In steps 4 and 5, application controller 130 and app 134 exchange handshake messages to establish a connection. In step 6, app 134 sends a user-selected activity to application controller 130. In step 7, app 134 sends a new skill model of the user-selected activity to application controller 130. The skill model identifies a particular action or a task (e.g., training) that the user will perform. The task may be a repetition of a combination of actions assigned by a coach.

In step 8, when an activity executable 132 (FIG. 1) for the activity has not been previously downloaded, application controller 130 requests the activity executable 132 from the cloud, i.e., from provider 202 over the Internet. Activity executable 132 includes a suit configuration (i.e., sensor configurations) for the user-selected activity and code for detecting actions, recognizing phases in the actions, and extracting metrics the phases. In step 9, provider 202 sends activity executable 132 to application controller 130 over the Internet.

In step 10, application controller 130 requests bus manager driver 126 to open a connection to motion capture suit 112. In step 11, bus manager driver 126 requests bus manager 128 to enable motion capture suit 112. In step 12, bus manager 128 informs bus manager driver 126 that motion capture suit 112 has been turned on. In step 13, bus manager driver 126 informs application controller 130 that motion capture suit 112 has been turned on. In step 14, application controller 130 sends the suit configuration for the activity to bus manager driver 126. In step 15, bus manager driver 126 forwards the suit configurations to bus manager 128, which configures motion sensors 114 accordingly. In step 16, bus manager 128 sends a ready status, suit diagnostic information, and an identification (ID) of motion capture suit 112 and sensors 114, 115 to bus manager driver 116. In step 17, bus manager driver 126 forwards the ready status, the suit diagnostic information, and the suit and sensor IDs to application controller 130. In step 18, application controller 130 sends the suit diagnostic information and the suit and sensor IDs to provider 202 for recording keeping and maintenance purposes.

In step 19, application controller 130 informs app 134 that application controller 130 is ready to capture motion data of the new activity. In step 20, application controller 130 runs activity executable 132 for the activity. In step 21, app 134 instructs application controller 130 to begin acquiring raw motion data from motion sensors 114 in motion capture suit 112 and on sports equipment 106. In some examples, application controller 130 also begin acquiring motion data from electronic device 120. In step 22, application controller 130 generates sparse geometric data streams from the raw data streams. Whereas the raw data streams contain motion data at a regular interval (e.g., 1,000 samples per second), the sparse data streams contain motion data when there is sufficient change from a prior value or sufficient time has passed from when the last value was recorded. In other words, one or more portions of a sparse data stream may contain motion data at irregular intervals. Also in step 22, activity executable 132 recognizes the action and its phases from the raw data streams and the sparse geometric data streams, extracts metrics from the phases, and sends the sparse geometric data streams and the metrics to app 134, which generates scores and the appropriate visual feedback to the user. The visual feedback may be an avatar, which is generated with skeletal model 150 in FIG. 3, illustrating the movement of the user.

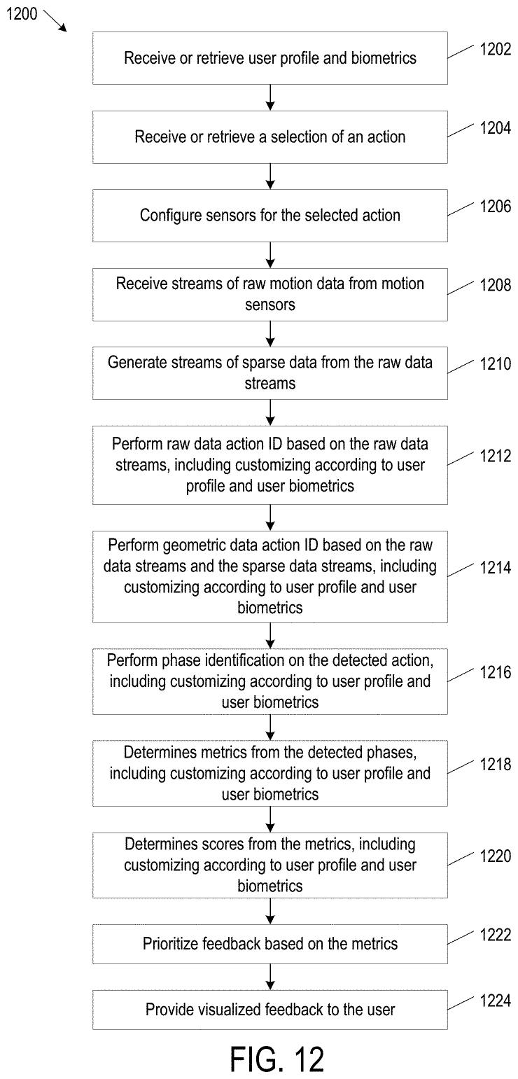

FIG. 12 is a flowchart of a method 1200 illustrating the operations of pod 118 (FIG. 1), more specifically application controller 130 (FIG. 1) and activity executable 132 (FIG. 1), in some examples of the present disclosure. Method 1200, and any method described herein, may be implemented as instructions encoded on a computer-readable medium that is to be executed by a processor a computing system. Method 1200, and any method described herein, may include one or more operations, functions, or actions illustrated by one or more blocks. Although the blocks are illustrated in sequential orders, these blocks may also be performed in parallel, and/or in a different order than those described herein. In addition, the various blocks may be combined into fewer blocks, divided into additional blocks, and/or eliminated based upon the desired implementation. Method 1200 may start with block 1202.

In block 1202, application controller 130 receives profile and biometrics of the user. Alternatively, application controller 130 retrieves from memory the user profile and the user biometrics. The user profile includes gender, date of birth, ethnicity, location, skill level, recent performance data, and health information. The user may input his or her profile through app 124 (FIG. 1), which transmits the information to application controller 130. The user biometrics include passive range of movement at each joint, active range of movement at each joint, strength indicator, anthropometric measurements, resting heart rate, and breathing rates. Biometric measurement may be taken at point of sale of pod 118 and recorded in the memory of the pod 118. Alternatively, the user may input his or her biometrics through app 124, which transmits the information to application controller 130. The user biometrics may be periodically updated by using app 124 or through provider 202 over the Internet. Block 1202 may be followed by block 1204.

In block 304, application controller 130 receives the user-selected activity and action from app 124. This corresponds to steps 6 and 7 of method 1100 in FIG. 11. Alternatively, application controller 130 retrieves the last selected activity and action from memory.

As mentioned previously, an action may be a physical skill, a technique of a skill, a variation of a technique, or a pose. A skill is defined as a movement that is part of an overarching activity (movement sequence, relate to medical or medical condition health) or a sport. For example, the golf swing is at the core of the game of golf. Nearly all golf swings have some aspects in common (e.g. using a club, a backswing, a downswing, a follow-through), which may be used to specify how to analyze a skill and, in particular, how to detect and analyze the skill to provide feedback.

The skill can be further specified according to a technique of the skill, the equipment being used, and a variation of the technique. For golf, the technique may be a type of shot, such as a drive, approach, chip, or putt, the equipment may be a type of golf club, such as a driver, 3 wood, or 7 iron, and the variation of the technique may be a shot shaping, such as straight, fade, draw, high, or low. For tennis, the technique may be a type of groundstroke, such as a forehand, backhand, volley, or serve, the equipment may be a type or size of tennis racket, such as stiffness, weight, or head size, and the variation of the technique may be a ball spin, such as topspin, flat, or slice. Specifying such information allows activity executable 132 to better identify when a user completes a skill.

Block 1204 may be followed by block 1206.

In block 1206, application controller 130 configures motion sensors 114 for detecting the action. This corresponds to step 14 in method 1100 of FIG. 11. For example, application controller 130 turns on a number of motion sensors 114 and sets their sampling rates for detecting the action. As described above, motion sensors 114 may be part of motion capture suit 112 (FIG. 1) and equipment 106. Block 1206 may be followed by block 1208.

In block 1208, application controller 130 receives time series of raw motion data (raw data streams) from corresponding motion sensors 114. Applicant controller 130 may also receive time series of biofeedback data (biofeedback data stream) from biofeedback sensor 115. Application controller 130 may further receive time series of additional motion data (additional motion data stream) from electronic device 120 (FIG. 1). Block 1208 may be followed by block 1210.

In block 1210, application controller 130 generates time series of sparse geometric data (sparse geometric data streams) from the raw data streams. Block 1210 may be followed by block 1212.

In blocks 1212 and 314, activity executable 132 performs action identification (ID). In block 1212, activity executable 132 performs raw data action ID to detect the action in time windows of the raw data streams. Activity executable 132 may use different thresholds and different motion sensors 114 or combinations of motion sensors 114 to identify different skills and different techniques. As this process uses raw motion data, it allows faster data processing than using more processed (fused) data. To improve detection, activity executable 132 may also use biofeedback data stream from biofeedback sensor 115 and the additional motion data stream from electronic device 120. Activity executable 132 may modify the identification process based on the user profile and the user biometrics received or retrieved in block 302. Block 1212 may be followed by block 1214.

In block 1214, activity executable 132 performs geometric data action ID to detect the action in the time windows identified in the raw data action ID. Activity executable 132 performs geometric data action ID based on the sparse geometric data streams in the identified time windows. To improve detection, activity executable 132 may also use the raw data streams from motion sensor 114, biofeedback data stream from biofeedback sensor 115 and the additional motion data stream from electronic device 120. Activity executable 132 may modify the identification process based on the user profile and the user biometrics received or retrieved in block 302. Block 1214 may be followed by block 1216.

In block 1216, activity executable 132 performs phase ID to detect phases of the detected action in the time windows identified in the geometric data action ID. Activity executable 132 performs phase ID based on the sparse geometric data streams in the identified time windows. To improve detection, activity executable 132 may also use the raw data streams from motion sensor 114, biofeedback data stream from biofeedback sensor 115 and the additional motion data stream from electronic device 120. Activity executable 132 may modify the phase identification based on the user profile and the user biometrics. Block 1216 may be followed by block 1218.

In block 1218, activity executable 132 determines metrics from the phases identified the phase ID. Activity executable 132 extracts the metrics from on the sparse geometric data in the identified phases. Activity executable 132 may also extra the metrics from on the raw data stream from motion sensor 114, biofeedback data stream from biofeedback sensor 115, and the additional motion data stream from electronic device 120. Activity executable 132 may modify the metrics being detected based on the user profile and the user biometrics. Block 1218 may be followed by block 1220.

In block 1220, app 124 determines scores based on the metrics received from pod 118. App 124 may modify the scoring based on the user profile and the user biometrics and according to preferences of the user or a coach. Details of block 320 are described later. Block 1220 may be followed by block 1222.

In block 1222, app 124 prioritizes feedback by applying weights to the scores, summing groups of the weighted scores to generate group summary scores, applying weights to the group summary scores, summing supergroups of the weighted group summary scores to generate supergroup summary scores, and generating a hierarchical structure based on the group summary scores and the supergroup summary scores. Block 1222 may be followed by block 1224.

In block 1224, app 124 uses the sparse geometric data streams of the detected action in identified windows to create and animate the avatar. App 124 may use the hierarchical structure of the scores to create a visual comparison between the avatar and an optimum performance. App 124 may enhance the visual comparison by indicating angle or distance notations based on the scores to highlight areas of interest. App 124 may also playback media based on the hierarchical structure of the scores.

Various other adaptations and combinations of features of the examples disclosed are within the scope of the invention. Numerous examples are encompassed by the following claims.

What is claimed is:

1: A motion capture suit, comprising:
a top or pants;
a sensor socket fixed to a portion of the top or the pants about a limb; and
a sensor inserted in the sensor socket, the sensor comprising a measurement device having at least two measurement axes offset about 45 degrees from a long axis of the limb.

2: The suit of claim 1, wherein:
the sensor comprises contact pins;
the sensor socket comprises contact sockets for receiving the contact pins; and
the contact pins and the contact sockets are arranged in a same pattern.

3: A motion capture suit, comprising:
a top or pants;
a sensor socket fixed to a portion of the top or the pants about a limb, the sensor socket comprising a socket base fixed to the portion of the top or the pants and a bezel rotatable around the socket base; and
a sensor attachable to the bezel.

4: The suit of claim 3, wherein:
the sensor comprises contact pins; and
the sensor socket comprises contact arcs for electrical connection with the contact pins.

5: The suit of claim 1, further comprising a sensor network comprising a chain of sensor sockets and sensors inserted into the sensor sockets, including the sensor inserted into the sensor socket.

6: The suit of claim 6, further the sensors include another sensor socket not fixed to any portion of the top or the pants, the other sensor socket comprising a clip for securing the other sensor socket to another piece of apparel.

7: The suit of claim 6, further comprising sensor networks, including the sensor network.

8: The suit of claim 1, wherein:
the sensor is part of a sensor set; and
the sensor set further comprises another sensor connected by a cable to the sensor.

9: The suit of claim 8, wherein the other sensor comprises an elastic loop or a clip.

10: The suit of claim 1, wherein the sensor includes an accelerometer and a gyroscope.

11: The suit of claim 10, wherein the sensor further includes a magnetometer.

12: The suit of claim 10, wherein the sensor further includes a microphone or a speaker.

13: The suit of claim 10, wherein the sensor further includes a light.

14: The suit of claim 10, wherein the sensor further includes a screen or an interactive display.

15: The suit of claim 10, wherein the sensor further includes a mobile device.

16: The suit of claim 10, wherein the sensor further includes a haptic feedback device.

17: The suit of claim 10, wherein the sensor further includes an environment sensor.

18: The suit of claim 10, wherein the sensor further includes a proximity sensor.
